(19) United States
(12) Patent Application Publication
(10) Pub. No.: US 2020/0383800 A1
(43) Pub. Date: Dec. 10, 2020

(54) A HOLDER FOR A FEMORAL KNEE COMPONENT

(71) Applicant: DEPUY IRELAND UNLIMITED COMPANY, RlNGASKIDDY (IE)

(72) Inventor: Conor LOWRY, LEEDS (GB)

(21) Appl. No.: 16/971,817

(22) PCT Filed: Feb. 21, 2019
(86) PCT No.: PCT/EP2019/054357

§ 371 Date: Aug. 21, 2020

(30) Foreign Application Priority Data
Mar. 5, 2018 (GB) 1803477.7

(51) Int. Cl. A61F 2/46

(52) U.S. Cl. CPC A61F 2/461; A61F 2002/4622

(57) ABSTRACT

There is provided a holder (100) for a femoral knee component (20), the holder comprising a pair of jaws which includes a first jaw (102) having a first femoral knee component engagement feature (110a) and a second jaw (104) having a second femoral knee component engagement feature (110b). The holder also includes a bridge (106) extending between the first jaw and the second jaw, the bridge being configured to allow the jaws to move between an open configuration for engaging with a femoral knee component and closed configuration for disengaging from the femoral knee component. A handle connection feature (112a) is also provided.

### TECHNICAL FIELD OF THE INVENTION

The invention relates to a holder for a femoral knee component for use during insertion of the femoral knee component into the femur, kits comprising the holder and methods of use thereof.

### BACKGROUND TO THE INVENTION

Joint arthroplasty is a well-known surgical procedure by which a diseased and/or damaged joint is replaced by a prosthetic component. A typical knee prosthesis includes a tibial tray, a femoral component, and a polymer insert or bearing positioned between the tibial tray and the femoral component.

A femoral impactor is placed on a femoral knee component and used to hammer the femoral implant into place. As shown in FIG. 1, typically the impactor includes a clamp adapted to engage an intercondylar feature of the femoral component, and at least one of the medial and lateral surfaces of the femoral component. The clamp is conventionally defined by a pair of sprung arms. A handle is peimanently secured to the clamp. The handle functions as an impaction rod.

Various problems are associated with the conventional femoral impactors. Problems include a tendency for mechanical failure due to the repetitive impaction forces applied to the multiple-component mechanism. Not only is mechanical failure of the femoral impactor during surgery frustrating to a surgeon, but safety and cost implications result. For example, there exists a possibility of a broken component (e.g. a spring) of the impactor being left within the wound. The delay in surgery as a replacement femoral impactor is found leads a prolonged surgical procedure, which can have health implications for the patient (e.g., the effects of a prolonged anaesthetic) and cost implications in terns of increased staff time and operating theatre times. There is also an associated increased cost as a new surgical instrument kit needs to be opened, and hence re-sterilised.

Typically due to the finite life of a femoral impactor, it is mandatory that it is taken out of service by a pre-determined expiry date. This again has cost implications, and requires monitoring of the surgical instrument inventory to ensure compliance.

In addition, ensuring complete sterilisation of the conventional femoral impactors can prove challenging. This is because bodily tissues and fluids may get trapped by the various movable components of the device or within inaccessible places.

There is therefore a need for an improved femoral impactor which addresses the problems associated with the conventional devices.

### SUMMARY OF THE INVENTION

According to a first aspect of the invention there is provided a holder for a femoral knee component, the holder comprising
a pair of jaws comprising
a first jaw having a first femoral knee component engagement feature,
a second jaw having a second femoral knee component engagement feature,
a bridge extending between the first jaw and the second jaw, the bridge being configured to allow the jaws to move between an open configuration for engaging with a femoral knee component and closed configuration for disengaging from the femoral knee component, and
a handle connection feature.

The femoral knee component holder of the present invention may also be referred as a femoral knee component inserter or a femoral knee component impactor.

A femoral knee component conventionally includes a medial femoral condyle, a lateral femoral condyle, and an intercondylar notch located therebetween.

Femoral knee component holders are releasably assembled onto the femoral knee component by the engagement of complementary elements provided on both components. For example, engagement features provided on the holder are complementary with engagement features typically provided on a medial surface of the medial femoral condyle and on the lateral surface of the lateral femoral condyle. Typically these engagement features are defined as a slot or a recess.

The first jaw may include an extension that terminates with a hook-shaped or other engagement feature adapted to engage a slot or other recess on one of the lateral or medial femoral condyles. The second jaw may include an extension that terminates with a hook-shaped or other engagement feature adapted to engage a slot or other recess on the other one of the lateral or medial femoral condyles.

Accordingly, the first femoral knee component engagement feature may be a hook-shaped element configured for engagement with a complementary slot or recess on the medial femoral condyle or lateral femoral condyle. The second femoral knee component engagement feature may also be a hook-shaped element configured for engagement within a complementary slot or recess on the other of the medial femoral condyle or lateral femoral condyle.

In order to attach the holder to the femoral knee component, a user moves the pair of jaws into the open configuration. The jaws are opened to such an extent that the holder can be positioned over and about the medial and lateral condyles. The jaws are then closed and the femoral knee component engagement features on the first and second jaws engage with complementary engagement features provided on the medial and lateral femoral condyles. The user then attaches a handle to maintain the pair of jaws in the open configuration.

In order to release the holder from the femoral knee component following impaction, a user removes the handle, and moves the jaws from the open configuration into the closed configuration, thereby disrupting the engagement between the femoral knee component engagement features on the holder and the complementary engagement features provided on the medial and lateral femoral condyles.

The holder may be made of a polymeric material. Advantageously, the polymeric material is a medical grade polymer and is suitable to be sterilised.

In some constructions, the holder is monolithic. The holder may be manufactured accordingly to methods known to the skilled artisan, which may include injection molding or 3-D printing.

The holder may disposable or capable of reuse (following re-sterilisation). The holder may be packaged as a single, disposable unit.

The ability to clean and re-sterilise the device is simplified when compared to conventional femoral impactors. This simplification results from the replacement of the intricate mechanical mechanisms associated with the conventional devices with a more simplified geometry, lacking any overlapping or inaccessible components.

In some constructions, each of the first jaw and the second jaw comprises a femoral knee component facing surface, in which the femoral knee component facing surface of the first jaw includes a contour that is generally complementary to the contour of a bearing surface of at least one of a lateral femoral condyle and a medial femoral condyle, and in which the femoral knee component facing surface of the second jaw has a contour that is generally complementary to a contour of a bearing surface of at least the other of the lateral femoral condyle and the medial femoral condyle.

In some constructions, the holder is symmetrical such that the first jaw and the second jaw are identical. Such a design of holder can be used with femoral knee component having symmetrical medial and lateral femoral condyles. The holder may be used in a first orientation such that the first jaw may be brought into contact with the medial femoral condyle and the second jaw may be brought into contact with the lateral femoral condyle. In addition, the holder may be used in a second orientation such that the first jaw may be brought into contact with the lateral femoral condyle and the second jaw may be brought into contact with medial femoral condyle.

In other constructions, the holder is asymmetrical such that the first jaw and the second jaw are non-identical. Such a holder is for use with a femoral knee component having asymmetrical medial and lateral femoral condyles. Such a design of holder can only be used in one orientation. The first jaw can only be brought into contact with the medial femoral condyle or the lateral femoral condyle, whereas the second jaw can only be brought into contact with the other of the medial femoral condyle and the lateral femoral condyle.

The femoral knee component facing surface of the first and second jaws may be a femoral knee component contacting surface.

In some constructions of the holder, the bridge includes a hinge. This hinge may be provided by a locally thinned section the polymeric material from which the first and second jaws are foimed.

The first end of the hinge may be connected to the femoral knee component facing surface of the first jaw and an opposing second end of the hinge is connected to the femoral knee component facing surface of the second jaw.

Optionally, the hinge comprises a femoral knee component facing surface having a contour that is generally complementary to the contour of an intercondylar notch provided between the lateral femoral condyle and the medial femoral condyle.

In some constructions, the femoral knee component facing surface of the hinge is a femoral knee component contacting surface.

In other constructions, a first end of the hinge is connected to a second jaw facing surface of the first jaw and an opposing second end of the hinge is connected to a first jaw facing surface of the second jaw.

In some other constructions of the holder, the bridge may include a post that extends from one of the first or second jaws and is configured for sliding receipt within a bore provided in the other of the first or second jaws. The post is configured to telescopically slide within the bore. This allows either an increase in the distance between the first and second jaws such that the pair of jaws is provided within the open configuration, or a decrease in the distance between the first and second jaws such that the pair of jaws is provided within the closed configuration.

In some constructions, the handle connection feature provided on the handle connection surface is a female member configured for removable receipt of a male member provided on the handle or vice versa.

In some constructions, the male handle connection feature is a generally T-shaped element, and the female handle connection feature is a complementary T-shaped slot.

Optionally, at least one handle connection feature is provided on the first jaw and at least one handle connection feature is provided on the second jaw.

Optionally, at least two spaced apart handle connection features are provided on the first jaw and at least two spaced apart handle connection features are provided on the second jaw.

Optionally, each of the first and second jaws comprises a handle connection surface which is opposed to the femoral knee component facing surface.

Optionally, at least one handle connection feature is provided on the handle connection surface of the first jaw and at least one handle connection feature is provided on the handle connection surface of the second jaw.

Optionally, at least two spaced apart handle connection features are provided on the handle connection surface of the first jaw and at least two spaced apart handle connection features are provided on the handle connection surface of the second jaw.

Handle connection features adjacent to a medial edge of the first jaw and the lateral edge of the second jaw primarily function to prevent the pair of jaws opening when the handle is attached. Handle connection features nearer to the lateral edge of the first jaw and the medial edge of the second jaw primarily function to transmit the impaction load from the handle, through the holder and to the lateral and medial femoral condyles during impaction.

The handle functions both as a handle and an impaction rod. The handle also controls rotation of the femoral component during impaction on the femur, and prevents the holder moving (e.g., flexing or sliding) from the open configuration to the closed configuration during impaction. Once the handle has been attached to the first and second jaws any relative movement of the first and second jaws is prevented. For example, in some constructions the hinge is retained in a flexed position. For example, any sliding movement of the post provided on one of the first and second jaws within the bore provided on the other of the first and second jaws is prevented.

According to a second aspect of the invention there is provided apparatus comprising a femoral knee component and a holder according to the first aspect of the invention releasably assembled thereon.

It is envisaged that in some constructions, a trial or a definite femoral knee component is packaged with the holder pre-assembled onto the implant component.

The surgeon may attach a handle to the implant/holder assembly before placing the implant on a resected femur, thereby forming an implant/holder/handle assembly.

The surgeon may attach a handle to the implant/holder assembly only after placing the implant on the resected femur.

According to a third aspect of the invention, there is provided a kit comprising a holder of according to the first aspect of the invention and a handle configured for releasable attachment thereto.

According to a fourth aspect of the invention there is provided a method of inserting a femoral knee component into a femur comprising the steps of:
using a holder according to the first aspect of the invention,
using a femoral knee component,
gripping the femoral knee component with the first femoral knee component engagement feature of the first jaw and with the second femoral knee component engagement feature of the second jaw,
positioning the femoral knee component on a resected femur and,
attaching a handle to the holder.

The step of attaching the handle to the holder may perfoimed prior to or after the positioning step.

The method further comprises impacting the femoral knee component on the femur.

The method further comprises disengaging the holder from the femoral knee component.

### BRIEF DESCRIPTION OF THE DRAWINGS

For a more complete understanding of the present invention and the advantages thereof, reference is now made to the following description taken in connection with the following drawings, in which:

FIG. 1 shows a conventional holder for a femoral knee component;

FIG. 2 shows a schematic of a first construction of the holder of the present invention;

FIG. 3 shows a schematic of a second construction of the holder of the present invention;

FIG. 4 shows a schematic of a handle for use with the first and second constructions of the holder;

FIG. 5 shows a schematic of the handle of FIG. 4 connected to the second construction of the holder as shown in FIG. 3.

FIG. 6 shows a schematic of a third construction of the holder of the present invention

### DETAILED DESCRIPTION OF THE INVENTION

Constructions of the present invention and the advantages thereof are best understood by referring to the following descriptions and drawings, wherein like numerals are used for like and corresponding parts of the drawings.

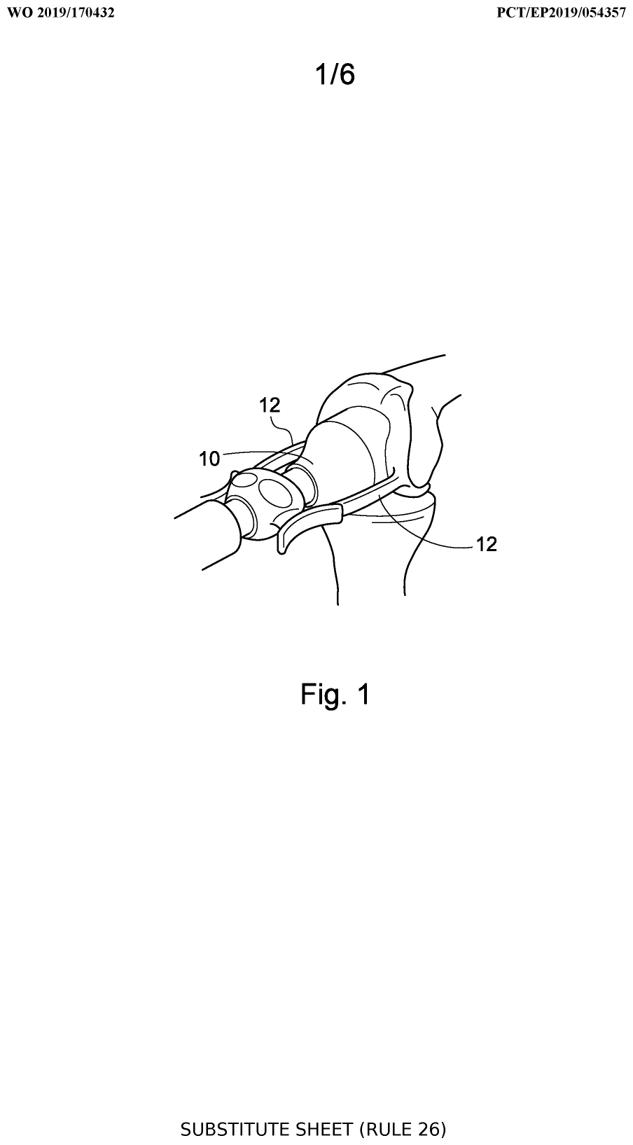

FIG. 1 shows a conventional holder 10 for a femoral knee component. The holder includes a pair of sprung-arms which include engagement features (not shown) configured for attachment to complementary engagement features (not shown) on the femoral knee component.

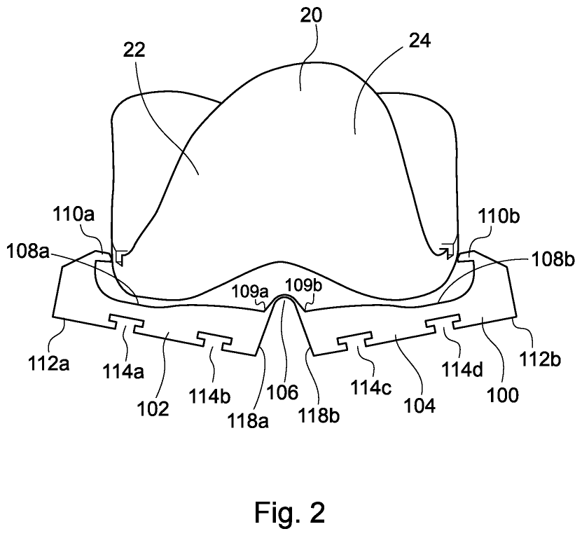

FIG. 2 shows a schematic of a first construction of the holder 100 of the present invention. The holder 100 is shown in open configuration retained about a femoral knee component 20. The holder has a pair of jaws composed of a first jaw 102 and a second jaw 104 connected via a bridge 106.

The first jaw 102 includes a femoral knee component facing surface 108a . The end portion of the jaw includes a femoral knee component engagement feature in the form of a hook-like element 110a.

In the construction shown, hook-like element 110a is configured for releasable receipt within a recess (not shown) on the lateral femoral condyle 22 of the femoral knee component.

The first jaw 102 also includes a handle connection face 112a . The handle connection face 112a includes a pair of spaced apart handle connection features 114a , 114b . In the construction shown, the handle connection features 114a , 114b are female and each is defined by a T-shaped slot configured for releasable receipt of a complementary T-shaped male connection feature provided on a handle.

The second jaw 104 includes a femoral knee component facing surface 108b . The end portion of the jaw includes a femoral knee component engagement feature in the form of a hook-like element 110b.

In the construction shown, hook-like element 110b is configured for releasable receipt within a recess (not shown) on the medial femoral condyle 24 of the femoral knee component.

The second jaw 104 also includes a handle connection face 112b . The handle connection face 112b includes a pair of spaced apart handle connection features 114c , 114d . In the construction shown, the handle connection features 114c , 114d are female and each is defined by a T-shaped slot configured for releasable receipt of a complementary T-shaped male connection feature provided on a handle.

The first jaw 102 and second jaw 104 are connected via a bridge 106. In the construction shown the bridge is defined by a hinge that extends from medial corner 109a of the femoral knee component facing surface 108a of the first jaw 102 to the lateral corner 109b of the femoral knee component facing surface 108b of the second jaw 104. In the construction shown, the holder is monolithic and made from a polymer. The hinge 106 is provided by a locally thinned portion of the polymer. The holder may be injection molded or 3D-printed.

The user places the holder in the open configuration in order to connect the holder to the femoral knee component 20. The user then connects a handle to the holder. The handle retains the holder in the open configuration and prevents the hinge inadvertently flexing during impaction. Such a flexing movement risks the holder being brought into the closed configuration, and thus disengaging the holder from the femoral knee component.

When the holder is to be disengaged from the femoral knee component 20 the user removes the handle and then moves the holder into the closed configuration. In the closed configuration the hook-like elements 110a , 110b are moved out of engagement from the complementary features (e.g., recesses) on the femoral knee component. The user moves the holder into the closed configuration by bringing the second jaw facing surface 118a of the first jaw 102 closer to, or indeed into contact with, the first jaw facing surface 118b of the second jaw 104.

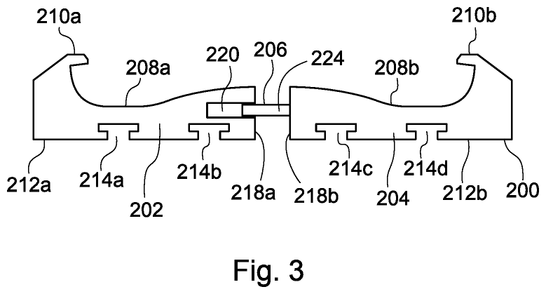

FIG. 3 shows a schematic of a second construction of the holder 200 of the present invention. The holder 200 can be retained in open configuration about a femoral knee component (not shown), in a similar manner to that shown in FIG. 2 in relation to the first construction of the holder 100. The holder 200 has a pair of jaws composed of a first jaw 202 and a second jaw 204 connected via a bridge 206.

The first jaw 202 includes a femoral knee component facing surface 208a . The end portion of the jaw includes a femoral knee component engagement feature in the foitu of a hook-like element 210a.

In the construction shown, hook-like element 210a is configured for releasable receipt within a recess (not shown) on the lateral femoral condyle (not shown) of the femoral knee component.

The first jaw 202 also includes a handle connection face 212a . The handle connection face 212 includes a pair of spaced apart handle connection features 214a , 214b . In the construction shown, the handle connection features 214a , 214b are female and each is defined by a T-shaped slot configured for releasable receipt of a complementary T-shaped male connection feature provided on a handle.

The second jaw 204 includes a femoral knee component facing surface 208b . The end portion of the jaw includes a femoral knee component engagement feature in the form of a hook-like element 210b.

In the construction shown, hook-like element 210b is configured for releasable receipt within a recess (not shown) on the medial femoral condyle (not shown) of the femoral knee component.

The second jaw 204 also includes a handle connection face 212b . The handle connection face 212b includes a pair of spaced apart handle connection features 214c , 214d . In the construction shown, the handle connection features 214c , 214d are female and each is defined by a T-shaped slot configured for receipt of a complementary T-shaped male connection feature provided on a handle.

The first jaw 202 and second jaw 204 are connected via a bridge 216. In the construction shown the bridge is defined by a recess 220 within the second jaw facing surface 218a of the first jaw 202 which is configured for sliding receipt of a post 224 that extends from the first jaw facing surface 218b of the second jaw 204.

In the construction shown, the first jaw 202 and second jaw 204 are distinct components made from a polymer. Each jaw of the holder may be injection molded or 3D-printed.

To connect the holder 200 to the femoral knee component (not shown), the user independently moves the first jaw 202 relative to the second jaw 204, and vice versa, so that the holder can extend about the lateral femoral condyle (not shown) and the medial femoral condyle (not shown), respectively and the hook-like elements 210a , 210b connect into respective complementary recesses on the lateral femoral condyle and the medial femoral condyle.

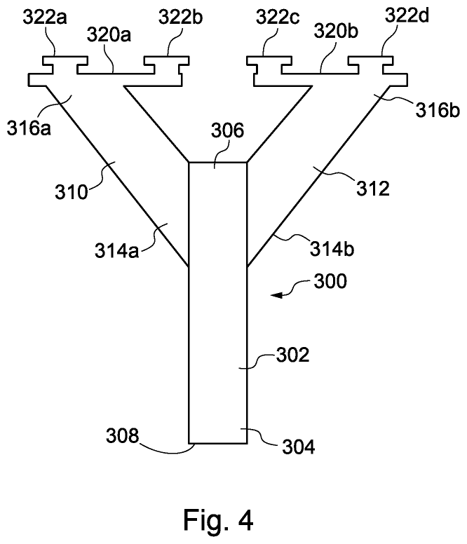

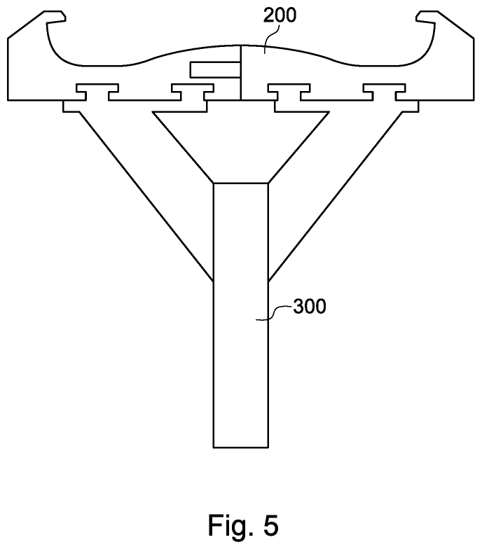

The user then connects a handle (see for example, FIG. 4) to the holder 200. This assembly is shown in FIG. 5. The handle retains the holder in the open configuration and prevents the handle inadvertently sliding into the closed configuration, in which it would disengage from the femoral knee component.

When the holder 200 is to be disengaged from the femoral knee component (not shown) the user removes the handle and then moves the holder into the closed configuration, in which the hook-like elements 210a , 210b are moved out of engagement with the complementary connection features on the femoral knee component. The user moves the holder into the closed configuration by sliding the first and second jaws towards each other, such that the second jaw facing surface 218a of the first jaw 202 is brought closer to or into contact with the first jaw facing surface 218b of the second jaw 204.

Referring now to FIG.4, there is shown a handle 300 suitable for use with both the first construction of the holder 100 or the second construction of the holder 200. The handle is generally Y-shaped. The handle includes a rod element 302 having a first end 304 and a second end 306. The first end is configured as an impaction surface 308. Each branch 310, 312 of the Y has a first end 314a , 314b attached to the rod element 302 and a second end 316a , 316b having a holder connection face 320a , 320b.

The holder connection face 320a of the first branch 310 includes a pair of spaced apart T-shaped male connection elements 322a , 322b.

The holder connection face 320b of the second branch 312 includes a pair of spaced apart T-shaped male connection elements 322c , 322d.

The handle is made of a medical grade metal. The handle can be re-sterilised. The handle may be reused, whilst the holder may be disposable.

Turning to FIG. 5, there is shown the handle 300 of FIG. 4 connected to the open configuration of the second construction of the holder of FIG. 3.

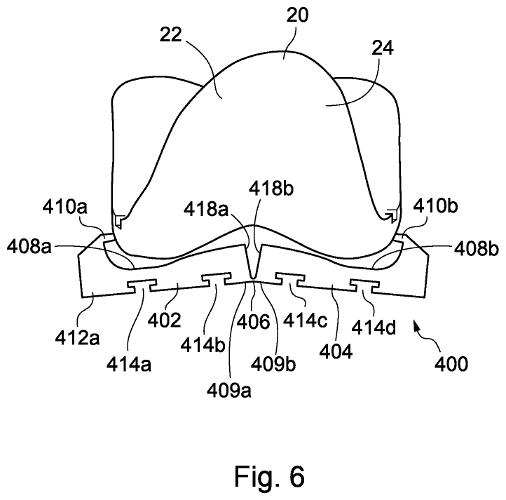

FIG. 6 shows a schematic of a third construction of the holder 400 of the present invention. The holder 400 is shown in open configuration retained about a femoral knee component 20. The holder has a pair of jaws composed of a first jaw 402 and a second jaw 404 connected via a bridge 406.

The first jaw 402 includes a femoral knee component facing surface 408a . The end portion of the jaw includes a femoral knee component engagement feature in the form of a hook-like element 410a.

In the construction shown, hook-like element 410a is configured for releasable receipt within a recess (not shown) on the lateral femoral condyle 22 of the femoral knee component.

The first jaw 402 also includes a handle connection face 412a . The handle connection face 412a includes a pair of spaced apart handle connection features 414a , 414b . In the construction shown, the handle connection features 414a , 414b are female and each is defined by a T-shaped slot configured for releasable receipt of a complementary T-shaped male connection feature provided on a handle.

The second jaw 404 includes a femoral knee component facing surface 408b . The end portion of the jaw includes a femoral knee component engagement feature in the form of a hook-like element 410b.

In the construction shown, hook-like element 410b is configured for releasable receipt within a recess (not shown) on the medial femoral condyle 24 of the femoral knee component.

The second jaw 104 also includes a handle connection face 412b . The handle connection face 412b includes a pair of spaced apart handle connection features 414c , 414d . In the construction shown, the handle connection features 414c , 414d are female and each is defined by a T-shaped slot configured for releasable receipt of a complementary T-shaped male connection feature provided on a handle.

The first jaw 402 and second jaw 404 are connected via a bridge 406. In the construction shown the bridge is defined by a hinge that extends from medial corner 409a of the handle connection face 412a of the first jaw 402 to the lateral corner 409b of the handle connection face 412b of the second jaw 404. In the construction shown, the holder is monolithic and made from a polymer. The hinge 406 is provided by a locally thinned portion of the polymer. The holder may be injection molded or 3D-printed.

The user places the holder in the open configuration in order to connect the holder to the femoral knee component 20. The user then connects a handle to the holder. The handle retains the holder in the open configuration and prevents the hinge inadvertently flexing during impaction. Such a flexing movement risks the holder being brought into the closed configuration, and thus disengaging the holder from the femoral knee component.

When the holder is to be disengaged from the femoral knee component 20 the user removes the handle and then moves the holder into the closed configuration. In the closed configuration the hook-like elements 410a , 410b are moved out of engagement from the complementary features (e.g., recesses) on the femoral knee component. The user moves the holder into the closed configuration by bringing the second jaw facing surface 418a of the first jaw 402 closer to, or indeed into contact with, the first jaw facing surface 418b of the second jaw 404.

Although particular constructions of the invention have been described, it will be appreciated that many modifications/additions and/or substitutions may be made within the scope of the claimed invention.

1-18. (canceled)

19. Apparatus comprising:
a femoral knee component and
a holder assembled thereon, in which the holder comprises;
a pair of jaws comprising:
a first jaw having a first femoral knee component engagement feature,
a second jaw having a second femoral knee component engagement feature,
a bridge extending between the first jaw and the second jaw, the bridge being configured to allow the jaw s to move betw een an open configuration for engaging with a femoral knee component and closed configuration for disengaging from the femoral knee component, and
a handle connection feature.

20. The apparatus of claim 19, in which each of the first femoral knee component engagement feature and the second femoral knee component engagement feature is hook-shaped and engageable with a slot of a corresponding medial or lateral surface of a femoral knee component.

21. The apparatus of claim 19, in which the holder comprises a polymeric material.

22. (canceled)

23. The apparatus of claim 19, in which the bridge includes a hinge.

24. The apparatus of claim 23, in which the hinge is provided by a locally thinned section of the polymeric material.

25. The apparatus of claim 19, in which each of the first jaw and the second jaw comprises a femoral knee component facing surface, in which the femoral knee component facing surface of the first jaw includes a contour that is generally complementary- to the contour of a bearing surface of at least one of a lateral femoral condyle and a medial femoral condyle, and in which the femoral knee component facing surface of the second jaw has a contour that is generally complementary to a contour of a bearing surface of at least the other of the lateral femoral condyle and the medial femoral condyle.

26. The apparatus of claim 25, in which the femoral knee component facing surface of each of the first and second jaws is a femoral knee component contacting surface.

27. The apparatus of claim 23, in which the hinge comprises a femoral knee component facing surface having a contour that is generally complementary to the contour of an intercondylar notch provided between the lateral femoral condyle and the medial femoral condyle.

28. The apparatus of claim 27, in which the femoral knee component facing surface of the hinge is a femoral knee component contacting surface.

29. The apparatus of claim 28, in which a first end of the hinge is connected to the femoral component facing surface of the first jaw and an opposing second end of the hinge is connected to the femoral component facing surface of die second jaw.

30. The apparatus of claim 23, in which a first end of the hinge is connected to a second jaw facing surface of the first jaw and an opposing second end of the hinge is connected to a first jaw facing surface of the second jaw.

31. The apparatus of claim 19. in which the bridge includes a post extending from one of the first or second jaw members, the post being configured for sliding receipt within a bore within the other of the first or second jaw members.

32. The holder of claim 19, in which the handle connection feature is a female member configured for removable receipt of a male member provided on the handle or vice versa.

33. The holder of claim 19, in which at least one handle connection feature is provided on the first jaw and at least one handle connection feature is provided on the second jaw.

34. The holder of claim 33, in which at least two spaced apart handle connection features arc provided on the first jaw and at least tw o spaced apart handle connection features are provided on the second jaw.

35. The holder of claim 25. in w hich each of the first and second jaws comprises a handle connection surface which is opposed to the femoral knee component facing surface, and in which at least one handle connection feature is provided on the handle connection surface of each jaw.

36. The apparatus of claim 19, in which the holder is disposable.

37. (canceled)

38. A method of inserting a femoral knee component into a resected femur comprising the steps of:
(a) using a holder which comprises
a pair of jaws comprising
a first jaw having a first femoral knee component engagement feature,
a second jaw having a second femoral knee component engagement feature.
a bridge extending between the first jaw and the second jaw, the bridge being configured to allow the jaws to move between an open configuration for engaging with a femoral knee component and closed configuration for disengaging from the femoral knee component, and
a handle connection feature,
using a femoral knee component,
(b) gripping the femoral knee component with the first femoral knee component engagement feature of the first jaw and with the second femoral knee component engagement feature of the second jaw,
(c) positioning the femoral knee component on the resected femur, and
(d) attaching a handle to the holder.

39. (canceled)

40. The method of claim 38, further comprising impacting the femoral knee component on the resected femur.

41. The method of claim 38, in which the method further comprises disengaging the holder from the femoral knee component.
